(19) United States
(12) Patent Application Publication
(10) Pub. No.: US 2011/0106228 A1
(43) Pub. Date: May 5, 2011

(54) BRAZING OF CERAMIC TO METAL COMPONENTS

(75) Inventors: Markus W. Reiterer, Plymouth, MN (US); Andrew J. Thom, Maple Grove, MN (US)

(73) Assignee: Medtronic, Inc., Minneapolis, MN (US)

(21) Appl. No.: 12/719,886

(22) Filed: Mar. 9, 2010

Related U.S. Application Data
(60) Provisional application No. 61/256,668, filed on Oct. 30, 2009.

(51) Int. Cl. A61N 1/375; B23K 1/20

(52) U.S. Cl. 607/115; 228/256

(57) ABSTRACT

A method for making a feedthrough assembly for an implantable electronic medical device comprises providing a metallic ferrule having an outer surface and an aperture defined by an inner lumen surface; providing an insulator, the insulator having a first surface and a second surface. At least one of the first surface and the second surface of the insulator includes a brazing region disposed thereon. The braze material is applied to the brazing region and the insulator is positioned within or around the metallic ferrule such that the positioned insulator brazing region and the metallic ferrule outer surface or inner lumen surface defines a braze gap. The braze gap has a width ranging between 10 μm to 50 μm. The feedthrough assembly is then heated at a temperature conducive to melt the braze material in the braze gap thereby forming a hermetic seal between the ferrule and said insulator.

### CROSS-REFERENCE TO RELATED APPLICATIONS

This application claims the benefit of U.S. Provisional Application No. 61/256,668, filed on Oct. 30, 2009. The entire disclosure of the above application is incorporated herein by reference.

### FIELD

The present teachings relate to electrical feedthrough assemblies of hermetically sealed implantable electronic devices.

### SUMMARY

The present teachings provide a method for making a feedthrough assembly. The method may include providing a metallic ferrule having an outer surface and a first aperture defined by an inner surface. An insulator may be provided in the first aperture, where the insulator has a first surface separated from the inner surface by a first braze gap, and a second surface defining a second aperture. A conductive element may be provided in the second aperture, where the conductive element is spaced from the insulator by a second braze gap. A braze material may then be applied in the first and second braze gaps and the assembly subsequently heated to braze the ferrule to the insulator, and to braze the conductive element to the insulator. The first and second braze gaps have a width that ranges between 10 and 50 μm, inclusive.

The present teachings also provide for a medical device. The medical device may include a housing and a connector module for connecting leads to electrical components internal to the housing. A feedthrough assembly located in the connector module connects the leads to the electrical components. The feedthrough may include a metallic ferrule, a conductive member, and an insulator disposed between the metallic ferrule and the conductive member. The insulator may be separated from the metallic ferrule by a first braze gap and may be separated from the conductive member by a second braze gap that are each filled with a braze material that heremetically seals the feedthrough assembly, wherein the first and second braze gaps have a width that ranges between 10 and 50 μm, inclusive.

Further areas of applicability will become apparent from the description provided herein. The description and specific examples in this summary are intended for purposes of illustration only and are not intended to limit the scope of the present teachings.

### DRAWINGS

The drawings described herein are for illustrative purposes only of selected embodiments and not all possible implementations, and are not intended to limit the scope of the present teachings.

FIG. 1 is a schematic diagram showing in cross-section view a feedthrough assembly according to various embodiments of the present teachings;

FIG. 2 is a schematic representation of a back-scattered electron image of the braze gap depicting the various phases and compositional arrangement of a titanium ferrule brazed with gold during brazing according to various embodiments of the present teachings;

FIG. 3 is an electron-probe microanalyzer graph depicting the relative amounts of the various intermetallic phases between a titanium ferrule and a gold braze during brazing between 700 and 1300° C. according to various embodiments of the present teachings.

Corresponding reference numerals indicate corresponding parts throughout the several views of the drawings.

### DETAILED DESCRIPTION

Exemplary embodiments are provided so that this disclosure will be thorough, and will fully convey the scope to those who are skilled in the art. Numerous specific details are set forth, such as examples of specific components, devices and methods, to provide a thorough understanding of embodiments of the present teachings. It will be apparent to those skilled in the art that specific details need not be employed, that exemplary embodiments may be embodied in many different forms and that neither should be construed to limit the scope of the present teachings. In some exemplary embodiments, well-known processes, well-known device structures and well-known technologies are not described in detail.

### Feedthrough Assemblies for Implantable Medical Devices

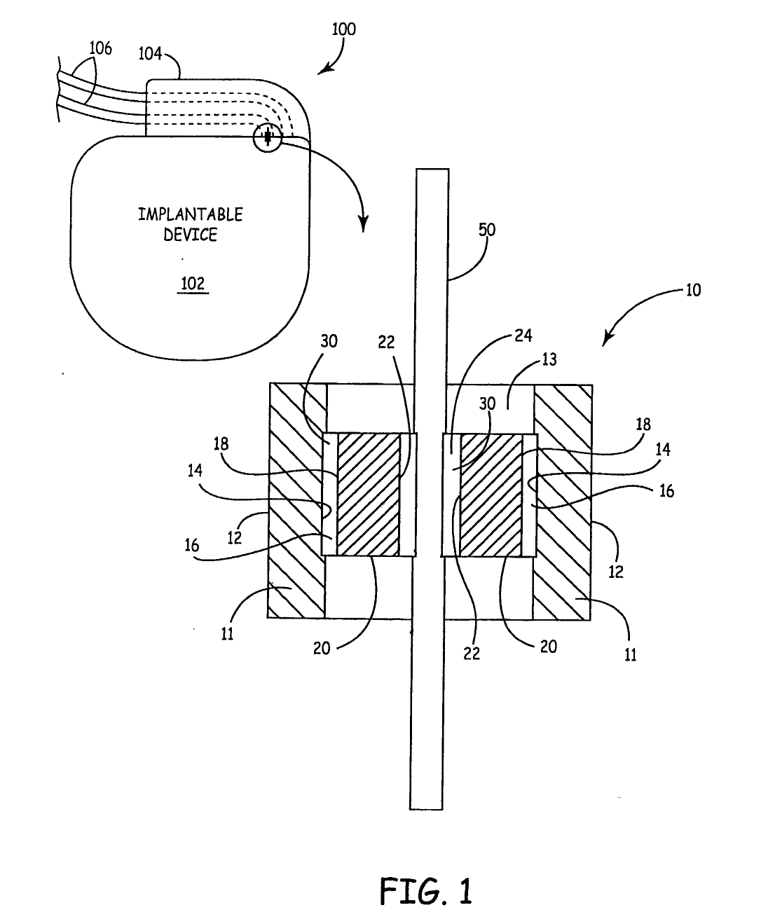

FIG. 1 illustrates an exemplary electronic implantable medical device 100 incorporating a feedthrough assembly 10 according to the present teachings. Medical device 100 may be any type of implantable device and, particularly, may be an implantable pulse generator for a cardiac pacemaker that provides electrical stimulation to an arrhythmic heart or neural tissue, an implantable defibrillator, an implantable cardioverter, an implantable cardiac pacemaker-cardioverter-defibrillator (PCD), an implantable chemical/biochemical sensor (e.g., a glucose sensor), an implantable drug, medicament or metabolite delivery device (e.g., an insulin pump), or an implantable medical device that performs in vivo diagnostic monitoring and telemetry. Regardless, medical device 100 generally includes a medical device housing 102 having a connector module 104 coupled thereto. Connector module 104 electrically couples various internal electrical components (not shown) located within medical device housing 102 to external operational and/or diagnostic systems (not shown) located distal to device 100 through use of leads 106. Electrical connection of leads 106 to the internal electrical components is accomplished through use of feedthrough assembly 10.

An exemplary feedthrough assembly 10 according to the present teachings may include a cylindrical ferrule 11, a conductive element 50 (e.g. a pin), and a cylindrical insulator 20. Ferrule 11 includes a ferrule outer surface 12 and a ferrule lumen surface 14 that defines an aperture 13. Ferrule 11 may be brazed to insulator 20 and, therefore, is separated from insulator 20 by a ferrule-insulator braze gap 16. Insulator 20 includes an insulator outer surface 18 and an insulator lumen surface 22. Insulator 20 may be brazed to conductive element 50 and, therefore, is separated from conductive element 50 by an insulator-conductive element braze gap 24. Braze gaps 16 and 24 are filled with braze material 30. While the exemplary embodiment in FIG. 1 shows a cross-section of a cylindrical insulator 20, a cylindrical ferrule 11, and a cylindrical conductive element 50, other shapes can be envisioned and the present teachings should not be limited thereto. Further, although only a single conductive element 50 is illustrated, it should be understood that feedthrough assembly 10 may include a ferrule 11 disposed about a plurality of conductive elements 50.

Moreover, other exemplary embodiments of feedthroughs are described in U.S. Pat. No. 4,678,868 issued to Kraska, et al. and entitled “Hermetic electrical feedthrough assembly,” in which an alumina insulator provides hermetic sealing and electrical isolation of a niobium electrical contact from a metal case. Further, for example, a filtered feedthrough assembly for implantable medical devices is also shown in U.S. Pat. No. 5,735,884 issued to Thompson, et al. and entitled “Filtered Feedthrough Assembly For Implantable Medical Device,” in which protection from electrical interference is provided using capacitors and Zener diodes incorporated into a feedthrough assembly. Other implantable feedthrough assemblies useful in the present teachings include those described in U.S. Pat. Nos. 7,164,572, 7,064,270, 6,855,456, 6,414,835 and 5,175,067 and U.S. Patent Application Publication No. 2006/0247714, all commonly assigned and all incorporated herein in their entireties.

Ferrule 11 may be formed of a conductive material. In some embodiments, the conductive material may be a metallic material including titanium, niobium, platinum, molybdenum, tantalum, zirconium, vanadium, tungsten, iridium, palladium, and any combination thereof. Ferrule 11 may have any number of geometries and cross-sections so long as ferrule 11 is an annular structure such as a ring with a lumen therein to hermetically seal insulator 20. In some embodiments, ferrule 11 may surround insulator 20 and provide ferrule lumen surface 14 to contact braze material 30 disposed in the ferrule-insulator braze gap 16 to form a hermetic seal.

Insulator 20 may be formed from a material including an inorganic ceramic material (e.g., sapphire), a glass and/or a ceramic-containing material (e.g., diamond, ruby, crystalline aluminum oxide, and zinc oxide), and an electrically insulative material. Insulator 20 may also be formed of liquid-phase sintered ceramics, co-fired ceramics, a high-temperature glass, or combinations thereof. Insulator 20 may also include a sputtered thin niobium or titanium-niobium coating at least at surfaces 18 and 22. Because the sputtered niobium coating is thin, the coating is not shown for illustration purposes. Insulator 20 is not limited to any particular configuration for use in feedthrough 10, so long as insulator 20 accommodates one or more electrically conductive elements 50.

Braze material 30 may be formed of a material such as gold. Other materials sufficient to braze ferrule 11 to insulator 20, and sufficient to braze insulator 20 to conductive element 50, however, are contemplated. For example, braze material 30 may include materials such as high purity gold, and gold alloys containing silver, copper, tin, and/or zinc without departing from the spirit and scope of the present teachings. The braze material can be reinforced with oxide, carbide, and nitride particles of refractory metals such as molybdenum, tungsten, hafnium, niobium, zirconium

Conductive element 50 may be formed of materials such as niobium, titanium, niobium-titanium alloy, titanium-6Al-4V alloy, titanium-vanadium alloy, platinum, iridium, molybdenum, zirconium, tantalum, vanadium, tungsten, palladium, nickel super alloy, nickel-chromium-cobalt-molybdenum alloy, and alloys, mixtures, and combinations thereof.

Feedthrough assembly 10 provides an electrical circuit pathway extending from the interior of hermetically-sealed device housing 102 to an external point outside housing 102 while maintaining the hermetic seal of the housing 102. The fluid tight hermetic seal is formed by metal braze 30 disposed in ferrule-insulator braze gap 16 and insulator-conductive element braze gap 24 formed between the insulator 20 and the ferrule 11 and between the insulator 20 and conductive element 50, respectively. A conductive path is provided through feedthrough 10 by conductive element 50, which is electrically insulated from housing 102.

According to the present teachings, there is a narrow requirement for widths of the braze gaps 16 and 24. Widths of ferrule-insulator braze gap 16 and insulator-conductive element braze gap 24 are controlled to tighter tolerances because if the dimensions are not closely controlled, the volume of the braze gap changes and only small variations of volume can be accommodated by brazing material 30, such as gold. If the gap volume is too small, oversized braze fillets may be formed and the gold braze can spill. Moreover, a convex shaped braze fillet may exert a strong tensile loading and promote delamination of braze 30. Further, if the gap volume is too big, the gaps 16 and 24 cannot be filled completely, and the feedthrough 10 will not pass performance requirements. Feedthrough assemblies 10 of the present teachings, therefore, have specified dimensional tolerances for braze gaps 16 and 24 such that ferrule-insulator braze gap 16 and insulator-conductive element braze gap 24 have a width ranging between about 10 μm to about 50 μm, inclusive. In some embodiments, widths of ferrule-insulator braze gap 16 and insulator-conductive element braze gap 24 may be 10 μm, or 20 μm, or 30 μm, or 40 μm, or 50 μm,

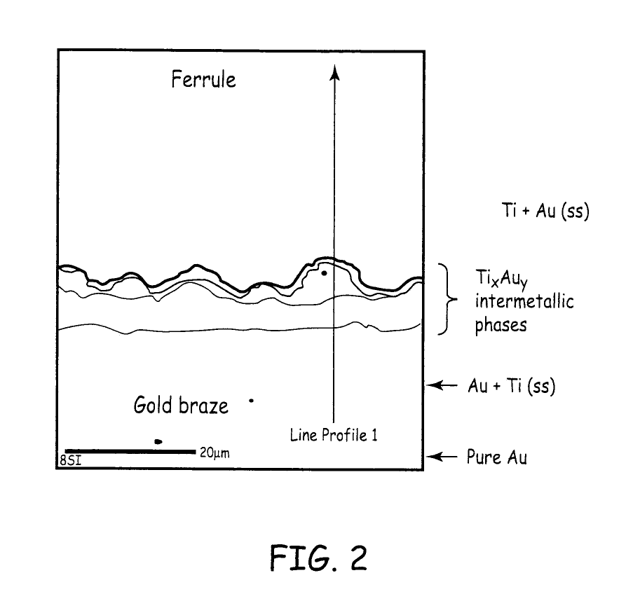

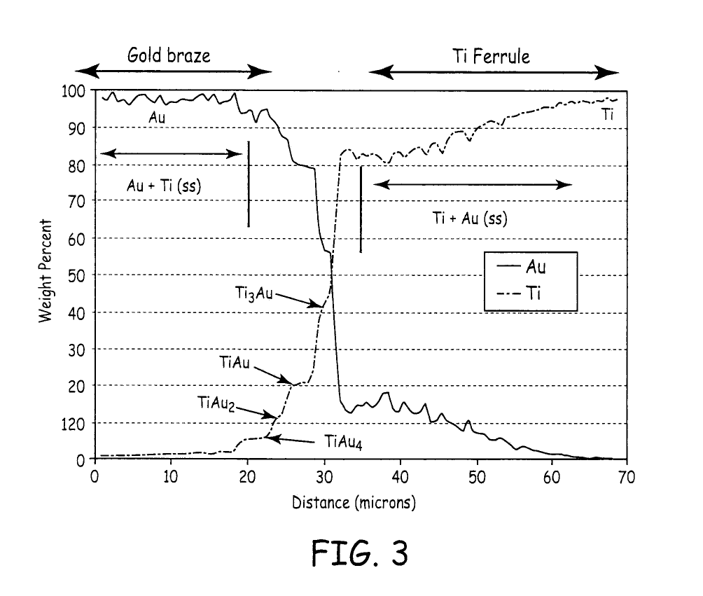

Feedthroughs 10 of the present teachings comprise a ferrule-insulator braze gap 16 and an insulator-conductive element braze gap 24 having widths ranging between about 10 μm to about 50 μm, inclusive, ensure that ferrule 11 and insulator 20 are hermetically adhered by brazing material 30. In this regard, during the brazing process, instantaneous alloying takes place between the niobium sputter coating disposed on insulator lumen surface 22 of insulator 20 and gold of braze material 30 adjacent pin 20, and the gold of braze material 30 and titanium of ferrule lumen surface 14. The concentration of niobium and titanium in the instantaneous alloying depends on the temperature schedule during brazing and on the widths of gaps 16 and 24 between insulator 20 and pin 50 and insulator 20 and ferrule 11. For example, during brazing, titanium and gold form a series of intermetallic compounds, and a gold mixed crystal phase field showing solid state (ss) solubility of roughly up to 6 mass % titanium as shown in FIG. 2 and FIG. 3.

When using a metallic ferrule 11 comprising titanium and a braze material 30 of gold, a gold-titanium solid state solution hardened mixture, and some of the titanium-gold intermetallic compounds, which can include TiAu₄, TiAu₂, TiAu and Ti₃Au. These instantaneously formed gold-titanium alloys are considerably stronger than pure gold, and contribute significantly to the mechanical performance of the brazed joint. This increase of strength is reached only if gap 16 is no larger than 50 μm to enable titanium from ferrule 11 to completely diffuse through braze gap 16, such that the entire braze gap 16 is occupied by the gold-titanium alloy. The local chemical composition of the braze 30 and the mechanical properties can be analyzed by microprobe and nano-indentation, respectively.

### Methods for Making a Feedthrough Assembly

With reference again to FIG. 1, feedthrough assembly 10 may be manufactured in the following exemplary manner. Insulator 20 can be inserted through ferrule 11 and then conductive element 50 can be inserted through insulator 20 such that gaps 16 and 24 between ferrule 11 and insulator 20, and between insulator 20 and conductive element 50, respectively, range between 10 μm to 50 μm, inclusive. Insulator 20 is hermetically bonded to ferrule 11 by placing braze material 30, for example, gold, in ferrule-insulator braze gap 16. Conductive element 50 is hermetically bonded to insulator 20 by placing braze material 30 in insulator-conductive braze gap 24. Feedthrough assembly 10 may then be heated at a temperature (e.g., 700° C.-1300° C.) able to melt braze material 30 in ferrule-insulator braze gap 16 and insulator-conductive element braze gap 24, thereby forming a hermetic seal between ferrule 11 and insulator 20, and between insulator 20 and pin 50 having instantaneously formed alloys that are considerably stronger than pure gold.

Insulators 20 of the present teachings may be made from ceramic materials or biocompatible, high-temperature co-fired alumina. Regardless, insulator 20 should have a very smooth insulator outer surface 18. Insulators 20 may be manufactured by applying tape casted green sheets of alumina mounted on frames. Through hole vias are punched, the vias can be filled with a platinum metal paste, and surface metallization may be screen printed. Individual sheets can be laminated, and subsequently fired. Next, dicing can be used to separate individual parts. Semi-circular ends can be manufactured by grinding. Infeed ultra-precision grinding can be used to obtain a partially effective insulator surface on a CNC grinder Absolute Grinding Company Inc., Cleveland, Ohio, USA, using a Studer S35 grinder according to manufacturer's specifications and instructions. Optionally, after surface inspection, cracks in surface 18 greater than 70 μm can be removed by ultrafine polishing. Insulator 20 may also be coated with a metallic film on the insulator outer surface 18 and insulator lumen surface 22 to enable wetting of braze material 30.

The insulator outer surface 18 can be polished using any commercial polishing machine, such as a polishing machine commercially available (for example, Struers RotoPol 35, Struers Inc., Cleveland, Ohio, USA) to the desired/specified surface quality (i.e., having no surface cracks at least at insulator outer surface 18 having a crack size greater than the critical flaw size ranging from about 30 μm to about 70 μm). Polishing of insulator 20, insulator outer surface 18, or insulator lumen surface 22 can be conducted at a cloth disc rotation of 150 rpm and a sample rotation of 40 rpm, respectively. Diamond suspensions of 3 μm and 0.05 μm (Kemet International Limited, Maidstone, Kent, UK) together with an ethanol-containing high-quality lubricant (DP-Lubrication Blue, Struers Inc., Cleveland, Ohio, USA) can be consecutively applied. The applied contacting force between insulator 20 and the cloth can vary from about 20N to about 80N. The polishing can be conducted for a period of time ranging from about 1 minute to about 60 minutes.

Outer surface 18 and insulator lumen surface 22 may be polished using a polishing procedure including rough polishing, intermediate polishing, and final polishing using 30 seconds of polishing time at each step. Three different abrasive papers with decreasing abrasiveness can be applied. For example, a 3 μm SiC paper with an air-cushion metal pad may be applied for rough polishing, 0.1 μm diamond paper with a metal-flat pad may be used for intermediate polishing, and 0.05 μm alumina paper with a rubber-flat pad may be used for final polishing. In each step, 91% volume isopropyl alcohol may be applied as a coolant. Sharp edges can further be rounded in a tumbling process. Such a polishing process achieves a surface quality having no cracks larger than the desired critical flaw size of 70 μm or less on the insulator outer surface 18 and insulator lumen surface 22 of the insulator 20 to be brazed. Results of the polishing steps can be verified using any one or more surface analysis tools, including confocal microscopy, electrical capacitance, electron microscopy and interferometer analysis. Cracks smaller than 11 μm may need to be removed if the crack size is greater than the critical flaw size for that particular insulator material when considering the insulator's acting stress.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method for making a feedthrough assembly, comprising:
providing a metallic ferrule having an outer surface and a first aperture defined by an inner surface;
providing an insulator in said first aperture, said insulator having a first surface separated from said inner surface by a first braze gap, and a second surface defining a second aperture;
providing a conductive element in said second aperture, said conductive element being spaced from said insulator by a second braze gap;
applying a braze material in said first and second braze gaps; and
heating the assembly to braze said ferrule to said insulator and to braze said conductive element to said insulator,
wherein said first and second braze gaps have a width ranging between 10 μm to 50 μm, inclusive.

2. The method of claim 1, wherein said insulator comprises at least one selected from the group consisting of a liquid-phase sintered ceramic, a co-fired ceramic, a high-temperature glass, and combinations thereof.

3. The method for making a feedthrough assembly for an implantable electronic medical device according to claim 2, wherein the insulator comprises a polycrystalline form of aluminum oxide.

4. The method of claim 1, wherein said braze material is gold and said ferrule is formed of titanium, and heating of the assembly forms intermetallic phases and a solid state solution alloy including the elements titanium and gold.

5. The method of claim 4, wherein said intermetallic compounds and gold titanium alloys have a hardness ranging from about 1 GPa to about 16 GPa.

6. The method of claim 1, wherein said insulator includes a thin niobium or titanium-niobium coating.

7. The method of claim 6, wherein said braze material is at least one selected from the group consisting of high purity gold, and gold alloys containing silver, copper, tin, and/or zinc, and heating of the assembly forms intermetallic compounds including niobium and gold.

8. The method of claim 1, further comprising heating the assembly to a temperature ranging from about 700° C. to about 1300° C. to braze said ferrule to said insulator and to braze said conductive element to said insulator.

9. A medical device comprising:
a housing;
a connector module for connecting leads to electrical components internal to said housing; and
a feedthrough assembly located in said connector module connecting said leads to said electrical components, said feedthrough including:
a metallic ferrule;
a conductive member; and
an insulator disposed between said metallic ferrule and said conductive member, said insulator being separated from said metallic ferrule by a first braze gap and being separated from said conductive member by a second braze gap each filled with a braze material that heremetically seals said feedthrough assembly, wherein said first and second braze gaps have a width ranging between 10 μm to 50 μm, inclusive.

10. The medical device of claim 9, wherein said insulator comprises at least one of liquid-phase sintered ceramic, a co-fired ceramic, a high-temperature glass, or combinations thereof.

11. The medical device of claim 10, wherein said insulator comprises a polycrystalline form of aluminum oxide.

12. The medical device of claim 9, wherein said braze material in said first braze gap includes intermetallic phases and a solid state solution alloy including the elements titanium and gold.

13. The medical device of claim 12, wherein said intermetallic compounds and gold titanium alloys have a hardness ranging from about 1 GPa to about 16 GPa.

14. The medical device of claim 9, wherein said insulator includes a thin niobium or titanium-niobium coating.

15. The medical device of claim 14, wherein said braze material in said second braze gap includes intermetallic compounds including niobium and gold.

16. The medical device of claim 9, wherein said housing is for one of an implantable pulse generator, an implantable defibrillator, an implantable cardioverter, an implantable cardiac pacemaker-cardioverter-defibrillator (PCD), an implantable chemical/biochemical sensor, and implantable drug delivery device
